(19) United States
(12) Patent Application Publication
(10) Pub. No.: US 2019/0183737 A1
(43) Pub. Date: Jun. 20, 2019

(54) MEDICAL OBJECT DISTRIBUTION SYSTEM AND METHOD

(71) Applicant: Fredrick M. Valerino, SR., Timonium, MD (US)

(72) Inventor: Fredrick M. Valerino, SR., Timonium, MD (US)

(21) Appl. No.: 16/220,786

(22) Filed: Dec. 14, 2018

Related U.S. Application Data
(60) Provisional application No. 62/598,982, filed on Dec. 14, 2017.

(51) Int. Cl. A61J 7/00; G16H 20/13

(52) U.S. Cl. CPC A61J 7/0076; G16H 10/60; G16H 20/13

(57) ABSTRACT

A pneumatic tubing system for distribution of medical objects in a medical environment without a dedicated carrier. The system comprises a plurality of headwall stations, a sending or receiving station, at least one pump, and at least one controller. Each headwall station is adapted to be installed in a headwall structure proximate to a patient bedside. The sending or receiving station is connected to each of the headwall stations via pneumatic tubing. The at least one pump is adapted to create pressure differentiation within at least a subset of the pneumatic tubing. The at least one controller is adapted to selectively activate the at least one pump to route a medical object between the sending or receiving station and one of the headwall stations within the pneumatic tubing.

### CROSS-REFERENCE TO RELATED APPLICATIONS

This application claims priority to U.S. Provisional Application Ser. No. 62/598,982, filed on Dec. 14, 2017, the entire contents of which is expressly incorporated herein by reference.

### BACKGROUND OF THE INVENTION

### Technical Field

Aspects of the embodiments relate to distribution of medical objects, such as medicines, and more specifically to systems, methods, and modes for automated distribution of medical objects directly to or from a headwall unit at a patient's bedside.

### Background Art

The practice of dispensing and delivery of medical objects at medical institutions, such as hospitals, is a time consuming process. Medications, for example, need to be prepared and transported in a secure environment while ensuring the safety of patients and hospital workers.

Pneumatic tube systems have been used in hospitals to transport medications from the pharmacy to the nursing station. Pneumatic delivery systems are used extensively for their rapid, efficient, and secure transportation of a wide variety of articles. Typical pneumatic tube systems comprise a sending station located at the pharmacy in communication with a plurality of receiving stations located throughout the hospital. When the doctor prescribes medication, the pharmacy fills the prescription and delivers it inside the carrier to a desired receiving station via the pneumatic tube system. To transport an object via conventional pneumatic tubing, the object needs to be first placed within a carrier, which is transported via the pneumatic tubing to a destination by air under either positive or negative pressure created by a blower or a fan. The interior of the closed tube and the outer dimension of the carrier form a seal so that the carrier can be propelled between pneumatic stations.

The nurse at the receiving station must open the carrier and determine to which patient at which location (e.g., a patient room, emergency room (ER), operating room (OR), or phlebotomy room) the nurse needs to deliver the prescription. The medications are sorted at the nursing station for delivery to the patients by nurses responsible for the patient. In many instances, however, the medications get mixed up due to frequent handling by numerous personnel. Secure storage at the nursing station may not be available as the medications await delivery by the nurses. Nurses delivering the medications may pick up the wrong medication to deliver. User error may occur during the identification of the room and patient resulting in miss-delivery of the prescription. Additionally, errors may occur as nurses may be delivering a plurality of medications to various rooms.

Accordingly, a need has arisen for systems, methods, and modes for quick, secure, and automated distribution of medical objects directly to or from a headwall unit at a patient's bedside.

### SUMMARY OF THE INVENTION

It is an object of the embodiments to substantially solve at least the problems and/or disadvantages discussed above, and to provide at least one or more of the advantages described below.

It is therefore a general aspect of the embodiments to provide systems, methods, and modes for quick, secure, and automated distribution of medical objects directly to or from a headwall unit at a patient's bedside.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Further features and advantages of the aspects of the embodiments, as well as the structure and operation of the various embodiments, are described in detail below with reference to the accompanying drawings. It is noted that the aspects of the embodiments are not limited to the specific embodiments described herein. Such embodiments are presented herein for illustrative purposes only. Additional embodiments will be apparent to persons skilled in the relevant art(s) based on the teachings contained herein.

### DISCLOSURE OF INVENTION

According to one aspect of the embodiments, a pneumatic tubing system is provided for distribution of medical objects in a medical environment without a dedicated carrier. The system comprises a plurality of headwall stations, a sending station, at least one pump, and at least one controller. Wherein each headwall station is adapted to be installed in a headwall structure proximate to a patient bedside, wherein each headwall station comprises an opening in communication with pneumatic tubing. Wherein the sending station is connected to each of the headwall stations via pneumatic tubing. Wherein the at least one pump is adapted to create pressure differentiation within at least a subset of the pneumatic tubing for transmitting a medical object from the sending station to one of the headwall stations. Wherein the at least one controller is in signal communication with the sending station and the at least one pump, wherein the controller is adapted to selectively activate the at least one pump to route a medical object from the sending station to one of the headwall stations within the pneumatic tubing.

According to an embodiment, at least one headwall structure comprises a structural frame and a decorative front panel, wherein at least one headwall station is adapted to be recessed within the structural frame, and wherein the decorative front panel comprises an opening to provide access to the at least one headwall station. According to another embodiment, at least one headwall structure comprises a headwall cabinet, wherein at least one headwall station is secured to the headwall cabinet, and wherein the headwall cabinet comprises an opening to provide access to the at least one headwall station.

According to an embodiment, at least one headwall station comprises a user interface, wherein the at least one headwall station comprises a door with a lock, wherein the user interface is adapted to receive user identifying information, wherein the at least one controller receives and authenticates the user identifying information from the at least one headwall station and upon successful authentication unlocks the door of the at least one headwall station. According to another embodiment, at least one headwall station comprises a user interface, wherein the user interface is adapted to receive prescription information and transmit the prescription information to the sending station.

According to an embodiment, the sending station comprises a plurality of inlets each comprising an opening in communication with pneumatic tubing, wherein each headwall station is associated with and connected to one of the inlets via an independent pneumatic tubing path, and wherein the system further comprises a plurality of the pumps, wherein each pump is associated with one of the headwall stations and is adapted to create pressure differentiation within an associated independent pneumatic tubing path. Each inlet may comprise a medical object guiding portion that tapers from a wider opening to a narrower opening to guide medical objects into a respective pneumatic tubing path.

According to an embodiment, the at least one controller is further adapted to: receive destination information from the sending station, identify an intended headwall station using the destination information, identify a pump associated with the intended headwall station from the plurality of pumps, and activate the identified pump to route a medical object from an inlet of the sending station associated with the identified headwall station to the identified headwall station. According to an embodiment, each inlet comprises a door adapted to close the inlet opening, wherein each door comprises a lock adapted to keep the door locked, wherein the at least one controller is further adapted to unlock the door of the inlet associated with the identified headwall station to automatically provide access to the identified headwall station via the associated inlet. The door may comprise a biasing mechanism adapted to bias the door to open, wherein upon being unlocked the door of the inlet associated with the identified headwall station is forced to an open position.

According to an embodiment, the medical object comprises an ID tag including destination information, wherein the sending station comprises an identifying tag reader adapted to read the ID tag of the medical object to retrieve the destination information. The destination information may comprise at least one of a room number or ID associated with a headwall station, a unique ID of a headwall station, a unique ID of a pump associated with a headwall station, and any combinations thereof.

According to another aspect of the embodiments, a pneumatic tubing system is provided for distribution of medical objects in a medical environment without a dedicated carrier comprising: a plurality of headwall stations, a sending station, a plurality of pumps, and at least one controller. Wherein each headwall station is adapted to be installed in a headwall structure proximate to a patient bedside, wherein each headwall station comprises an opening in communication with pneumatic tubing. Wherein the sending station comprises a plurality of inlets each associated with and connected to one of the headwall stations via an independent pneumatic tubing path. Wherein each pumps is adapted to create pressure differentiation within at least one independent pneumatic tubing path. Wherein the at least one controller is in signal communication with the sending station and the at least one pump. Wherein the controller is adapted to: receive destination information from the sending station, identify an intended headwall station using the destination information, identify a pump associated with the intended headwall station from the plurality of pumps, and activate the identified pump to route a medical object from an inlet of the sending station associated with the identified headwall station to the identified headwall station.

According to another aspect of the embodiments, a pneumatic tubing system is provided for disposal of medical objects in a medical environment without a dedicated carrier. The system comprises a plurality of headwall stations, a receiving container, at least one pump, and at least one controller. Wherein each headwall station is adapted to be installed in a headwall structure proximate to a patient bedside, wherein each headwall station comprises an opening in communication with pneumatic tubing. Wherein the receiving container is connected to each of the headwall stations via pneumatic tubing. Wherein the at least one pump is adapted to create pressure differentiation within at least a subset of the pneumatic tubing for transmitting a medical object from one of the headwall stations to the receiving container. Wherein the at least one controller is in signal communication with the plurality of headwall stations and the at least one pump, wherein the controller is adapted to selectively activate the at least one pump to route a medical object from one of one of the headwall stations to the receiving container within the pneumatic tubing.

According to an embodiment, each headwall station comprises a medical object guiding portion tapering from a wider opening to a narrower opening in order to guide the medical object into the pneumatic tubing.

According to an embodiment, the system further comprises a plurality of receiving containers connected to the plurality of the headwall stations via pneumatic tubing, wherein the at least one controller selectively routes the medical object from one of the headwall stations to a selected one of the receiving containers. According to an embodiment, the system further comprises a plurality of pumps each associated with one of the receiving containers, wherein the at least one controller selectively activates at least one pump of a desired receiving container to route the medical object from one of the headwall stations to a selected one of the receiving containers. According to another embodiment the system comprises a diverter, wherein the at least one controller is adapted to activate the diverter to route the medical object from one of the headwall stations to a selected one of the receiving containers. According to an embodiment, each receiving container comprises a sensor adapted to detect when the receiving container is full, wherein the at least one controller designates a first container from the plurality of receiving containers to receive disposed medical objects and routes the medical objects to the first designated receiving container, wherein when the sensor of the first designated receiving containers indicates that the first designated receiving container is full, the at least one controller designates a second receiving container from the plurality of containers to receive disposed medical objects and routes the medical objects to the second designated receiving container.

### BRIEF DESCRIPTION OF THE DRAWINGS

The above and other objects and features of the embodiments will become apparent and more readily appreciated from the following description of the embodiments with reference to the following figures. Different aspects of the embodiments are illustrated in reference figures of the drawings. It is intended that the embodiments and figures disclosed herein are to be considered to be illustrative rather than limiting. The components in the drawings are not necessarily drawn to scale, emphasis instead being placed upon clearly illustrating the principles of the aspects of the embodiments. In the drawings, like reference numerals designate corresponding parts throughout the several views.

### BRIEF DESCRIPTION OF THE SEVERAL VIEWS OF THE DRAWING

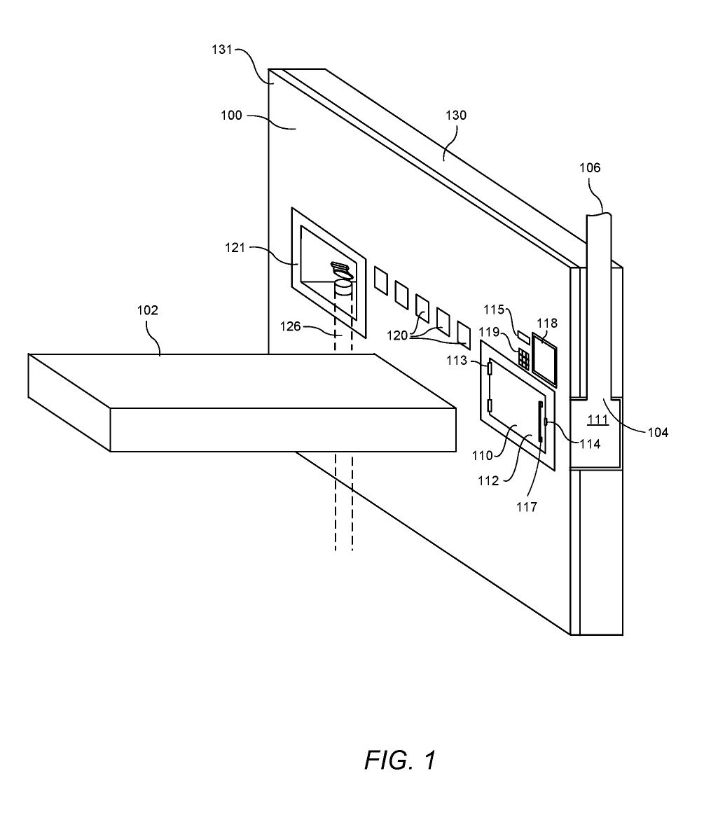

FIG. 1 illustrates a perspective view of a headwall with a plurality of headwall stations for distribution of medical objects according to an illustrative embodiment.

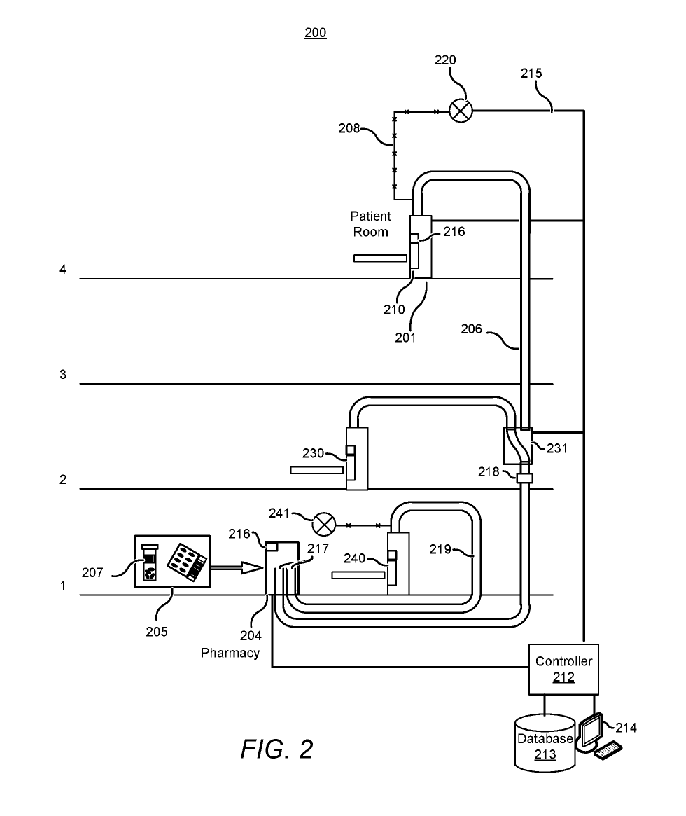

FIG. 2 shows a diagram illustrating a pneumatic tubing system for distribution of medical objects between a pneumatic station and at least one headwall station according to an illustrative embodiment.

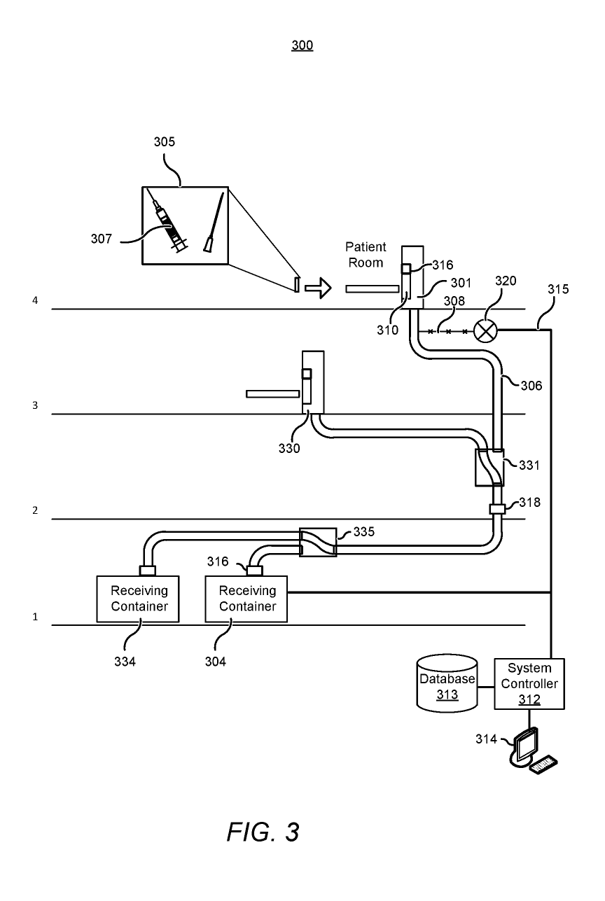

FIG. 3 shows a diagram illustrating a pneumatic tubing system for disposing medical objects, such as sharps, directly from a headwall station according to an illustrative embodiment.

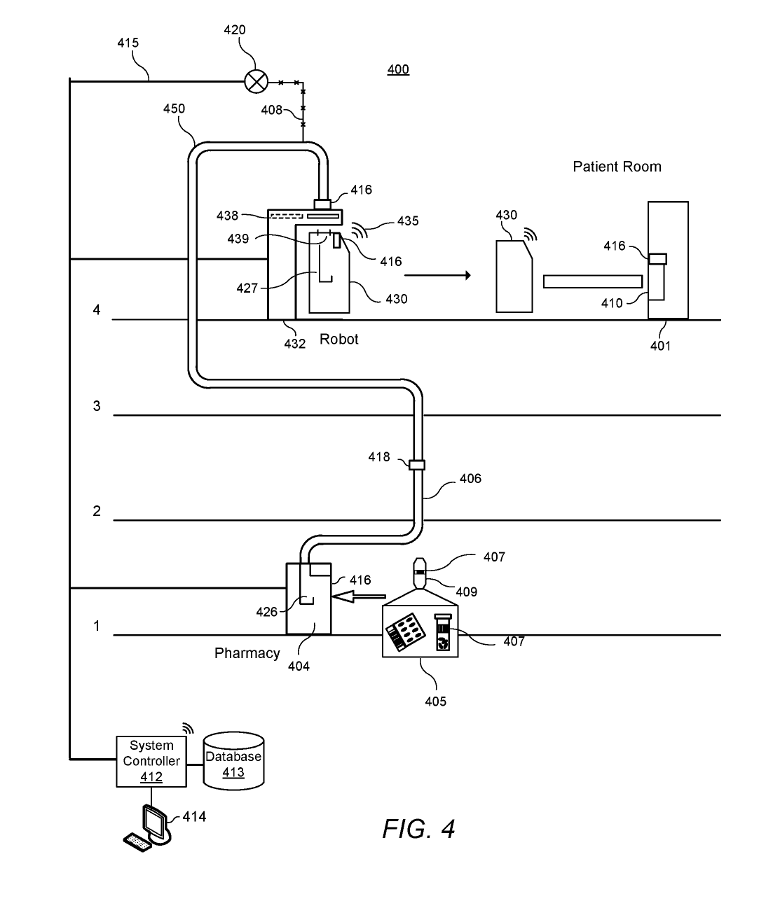

FIG. 4 shows a diagram illustrating an automated distribution system comprising a pneumatic tubing system and a robot for distribution of medical objects between a pneumatic station and a headwall station according to an illustrative embodiment.

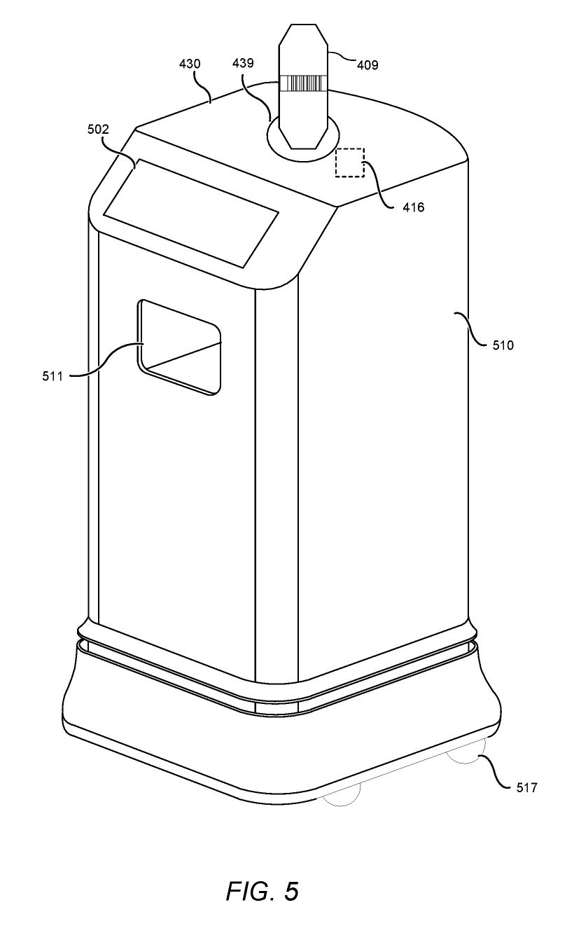

FIG. 5 illustrates a rear perspective view of a robot used in the automated distribution system according to an illustrative embodiment.

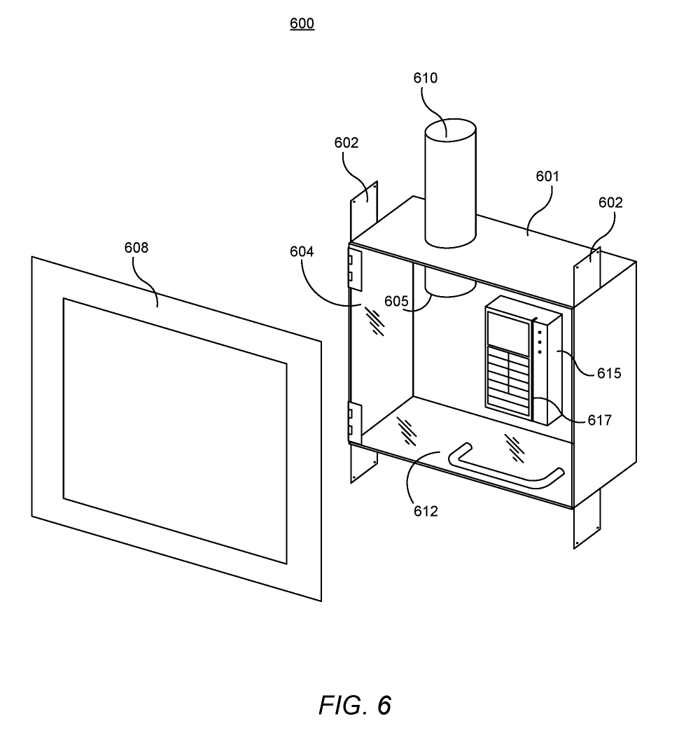

FIG. 6 illustrates a front perspective view of a medical object distribution headwall station according to an illustrative embodiment.

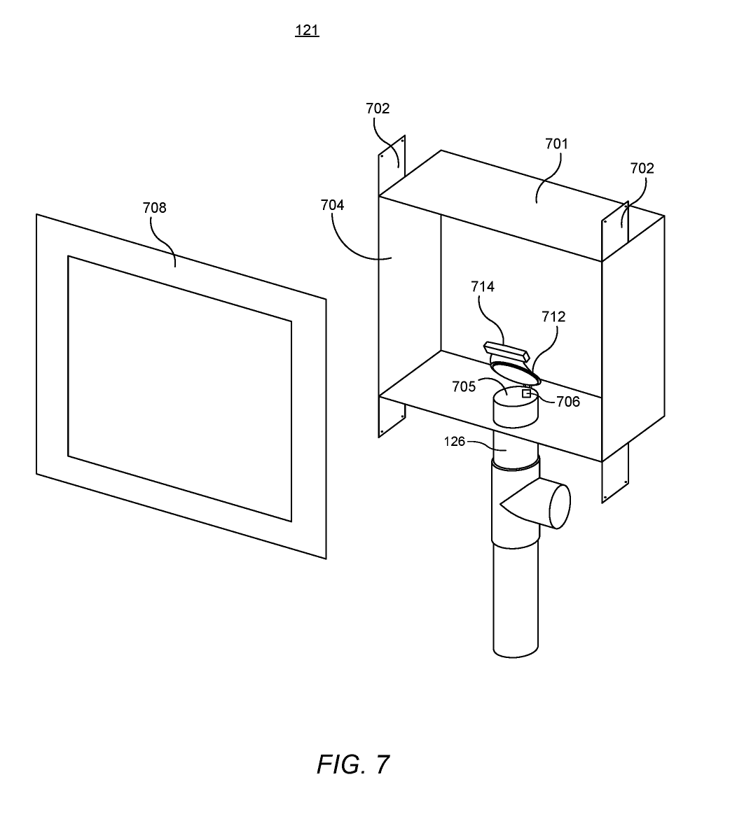

FIG. 7 illustrates a front perspective view of a medical object disposing headwall station according to an illustrative embodiment.

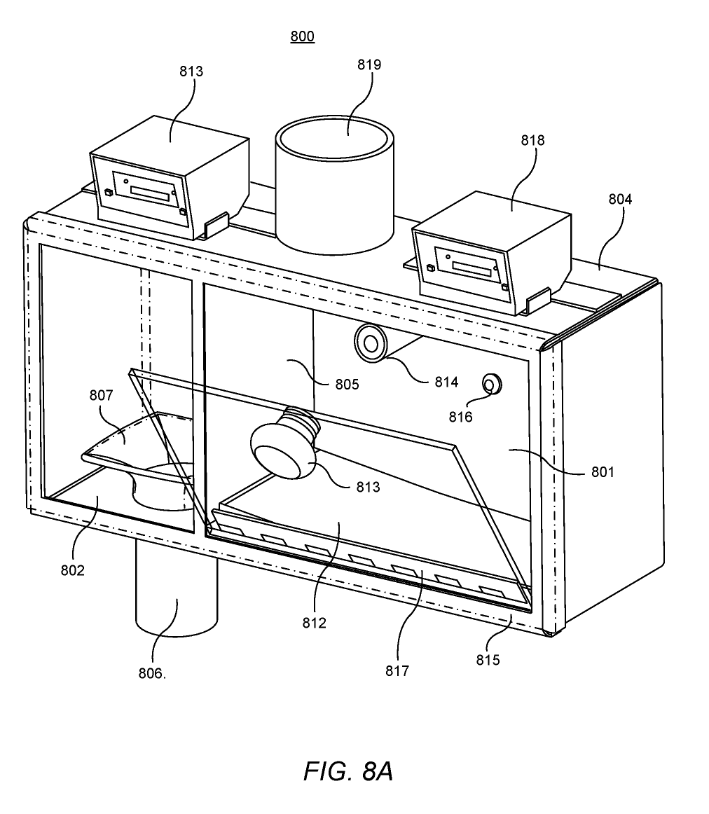

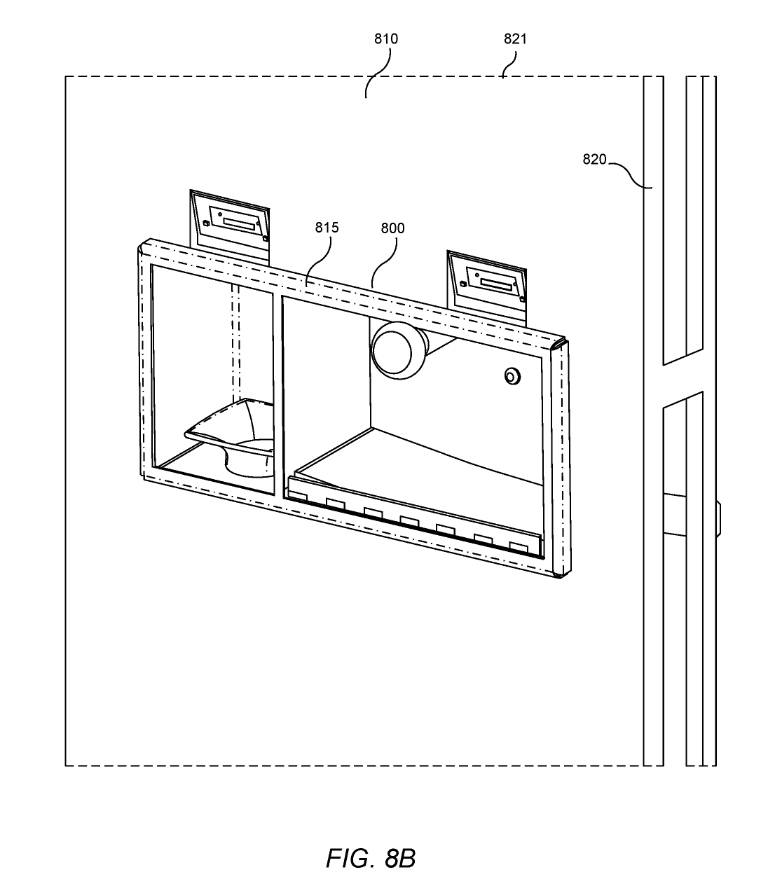

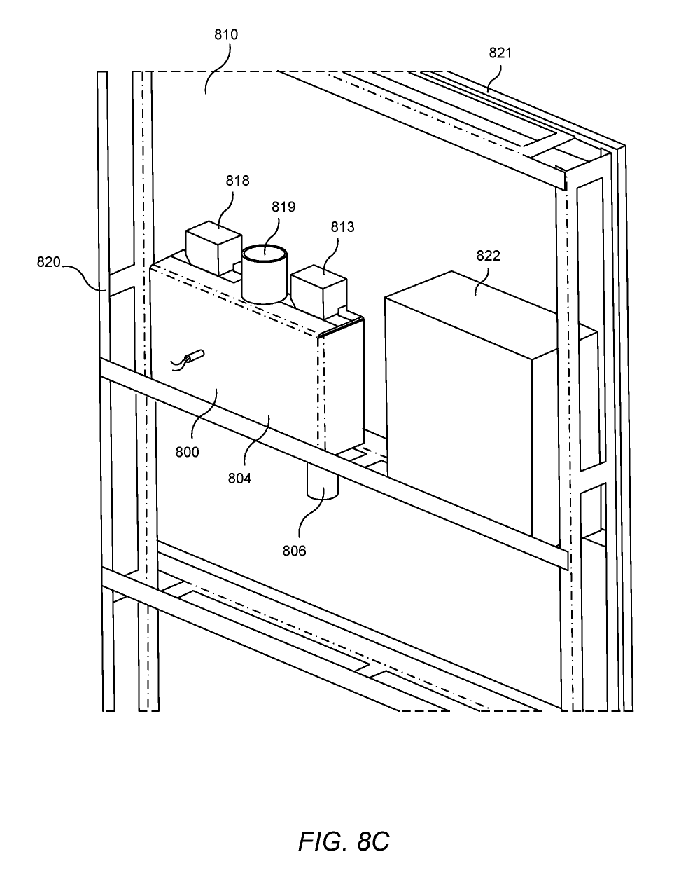

FIG. 8A illustrates a front perspective view of a headwall station comprising both a medical object receiving and dispensing portion or bin and a medical object disposal portion or bin according to an illustrative embodiment.

FIG. 8B illustrates a front perspective view of the headwall station in FIG. 8A installed in a headwall according to an illustrative embodiment.

FIG. 8C illustrates a rear perspective view of the headwall station in FIG. 8A installed in the headwall according to an illustrative embodiment.

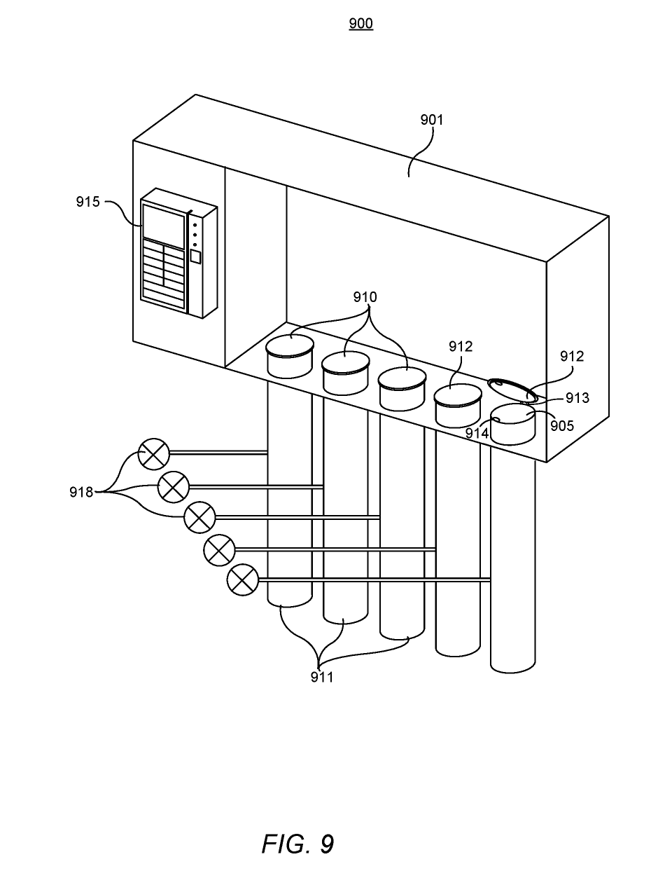

FIG. 9 illustrates a front perspective view of a medical object sending station according to an illustrative embodiment.

### DETAILED DESCRIPTION OF THE INVENTION

The embodiments are described more fully hereinafter with reference to the accompanying drawings, in which embodiments of the inventive concept are shown. In the drawings, the size and relative sizes of layers and regions may be exaggerated for clarity. Like numbers refer to like elements throughout. The embodiments may, however, be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” on “in an embodiment” in various places throughout the specification is not necessarily referring to the same embodiment. Further, the particular feature, structures, or characteristics may be combined in any suitable manner in one or more embodiments.

The different aspects of the embodiments described herein pertain to the context of systems, methods, and modes for automated distribution of medical objects directly to or from a headwall unit at a patient's bedside, but is not limited thereto.

Referring to FIG. 1, there is shown a perspective view of a headwall 100, or headboard, with a headwall station 110 and a headwall station 121 for distribution of medical objects according to an illustrative embodiment. Headwalls are usually placed at a head of a patient bed 102 in intensive care units (ICU) or in most critical care and deeply aggressive patient care areas. Headwalls are architectural structures that merge medical service equipment 120 to increase efficiency. Such medical service equipment 120 may comprise electrical and medical gas connections, such as medical gas outlets, power supplies, emergency electrical receptacles, as well as storage cabinets, monitor support, nurse calling equipment, or the like. Headwall 100 typically comprises a structural frame 130 and at least one decorative front panel 131 with cutouts or openings to provide access to the medical service equipment 120. The pneumatic systems according to the present embodiments provide for unattended or automated distribution of medical objects in a secure environment directly to or from the headwall 100 at the patient's bedside. According to one embodiment, headwall station 110 may be utilized in the pneumatic tubing system as an interface to the pneumatic tubing 106 to receive medical objects, such as medicines. According to a further embodiment, headwall station 121 may be utilized in the pneumatic tubing system as an interface to pneumatic tubing 126 to dispose medical objects, such as sharps. Medical object distribution, such as medicines or sharps, should be accessible adjacent to the patient. The present embodiments provide access to the automated medical distribution system by placing access points, such as headwall stations 110 and 121, directly at a patient headwall 100, which is conveniently placed adjacent to the patient bedside. As a result, medical objects can be directly delivered to the patient's bedside, and/or automatically disposed from the patient's bedside, depending on the implementation of the system.

In patient rooms where headwalls, such as headwall 100, are not utilized, headwall stations 110 and/or 121 may be mounted within a dedicated headwall receiving cabinet placed adjacent to the patient bedside. The headwall receiving cabinet may comprise an enclosed structure for housing the headwall stations 110 and/or 121 with an opening to provide access to the headwall station.

According to various embodiments, a single headwall station may be present by the patient's bedside, or a plurality of headwall stations, such as 110 and 121, may be positioned at the headwall 100 adjacent to the patient for different purposes. For example, headwall station 110 may be used to receive medical objects from a pharmacy or supply areas located within the hospital via pneumatic tubing 106. According to one embodiment, medical objects directly travel within pneumatic tubing 106 without any dedicated carriers. According to another embodiment, medical objects may be placed inside a cylinder or a carrier, which travels within pneumatic tubing 106. Such medical objects may include, but not limited to, medicines, such as oral, topical, or suppository pharmaceutical drugs provided in blister packs or small bottles, intravenous (IV) bags, blood unit bags, or similar solution bags, as well as small medical devices or supplies, such as sterile items, thermometers, bandages, dressings, or the like, used to diagnose, cure, treat, or prevent disease. According to another embodiment, the headwall station 110 may be limited to receiving routine pharmaceutical medications, such as blister packs of acetaminophen, ibuprofen, or the like.

Placing a headwall station 110 at each patient's bedside may save hospitals valuable space by providing safe and convenient storage of medical objects, such as medicines, prior to administration. No medical objects need to be stored at the nursing station or at the pharmacy. The headwall station 110 also enables the isolation of medical objects between patients, reducing errors of administering drugs to the wrong patient and improving patient security. The system also relieves hospital personnel and professionals of unnecessary travel by removing duplication of movement by personnel, if for example a nurse has forgotten to bring the medication to the patient's room for administration. Furthermore, the architecture of the pneumatic tubing system allows for the creation of a complete chain of custody of a medical object from the pharmacy and/or the nursing station to the patient.

According to another embodiment, headwall station 110 at patient's headwall 100 may be used to transmit medical objects, such as patient documentation or files, prescriptions, blood samples, viral samples, or other biological or chemical matter obtained from the patient, from the headwall station 110 to other areas of the hospital, for example to laboratories or to pharmacies within the hospital. For example, immediately after taking a blood sample at a patient's bedside, a nurse may use the headwall station 110 to transmit the blood sample to a laboratory via pneumatic tubing 106.

Headwall station 121 may be used for automatic and safe disposal of used medical objects, such as sharps, from a location directly adjacent to the patient to a secured central disposal location via pneumatic tubing. Sharps may include but not limited to needles, syringes with and without needles, hypodermic and tubing, acupuncture needles, suture needles, tubing with needles, scalpel blades, blood vials, exposed ends of dental wires, broken glass or capillary tubes, culture dishes and slides, lancets, pipettes, root canal files, trauma scene waste that can cut, slice or pierce, or the like. According to as aspect of the present embodiments, the sharps may be disposed without any dedicated pneumatic carrier. Instead, sharps may be directly transported within the pneumatic tubing 126. Accordingly, immediately after the sharps are used in a procedure, the user may dispose of the sharps directly via the headwall stations 121 located directly adjacent to the patient bedside. The sharps are then quickly transported to a remote receiving station. Since the sharps are immediately disposed of without the use of any containers or carriers, there is no danger that the container or the carrier may get overfilled. Moreover, no individual carriers or containers need to get loaded at the headwall station 121 or emptied every time the receiving station receives a delivery of sharps. Accordingly, the sharps disposal system of the present embodiments speeds up the disposal process and effectively reduces the extent a user handles the sharps, significantly reducing the risk of accidental needle-stick injury.

According to various embodiments, the headwall station 110 may comprise a housing 111 recessed in the headwall 100 behind the decorative front panel 131 of the headwall 100. The decorative front panel 131 may comprise an opening to provide access to the headwall station 110. Headwall station 110 may be configured and sized to fit and to be supported by the structural frame 130 of the headwall 100. The front face of the housing 111 may include an opening in communication with opening 104 that mates with a terminal end of the pneumatic tubing 106. The opening on the front face of the housing 111 may comprise a door 112 configured for closing the front of the housing 111 to restrict access to the headwall station 110 by unauthorized users. The door 112 may comprise at least one spring loaded hinge 113 that forces the door 112 shut. The door 112 may comprise a handle 117 that may be pulled by the user to open the door 112. When the handle is released, the spring loaded hinge 113 will force the door 112 to close. The door 112 may further comprise a see-through window through which a user, such as a nurse, can observe contents within the headwall station 110. The door 112 may also comprise a lock 114, such as an electromagnetic lock, that keeps the door 112 closed. Other types of locks may also be utilized, such as a key lock.

According to an embodiment, the headwall station 110 may further comprise an identifying tag reader 115 for reading ID tags of the user, or ones attached to the medical objects or its container, packaging, or carrier. The front face of the headwall station 110 may further comprise a user interface such as a touch-screen 118 or a keypad 119 for receiving user input. The user interface may be utilized to enter security information (e.g., a personal identification number (PIN)) for a user of the headwall station 110. User identifying information received by scanning a user ID tag via identifying tag reader 115, or security information received via the user interface 118, may be used for tracking purposes of the medical objects, or in another embodiment, may be used for unlocking the lock 113 of the door 112 to allow the user to open the door 112. The user interface may in addition may be used to enter destination information of medical objects that need to be sent from the headwall station 110. Furthermore, a user may enter prescription information using the user interface, which may be sent to the pharmacy.

Referring to FIG. 6, there is shown another embodiment of headwall station 600 for distribution of medical objects. Headwall station 600 may comprise housing 601 adapted to be recessed in a headwall of a hospital. Housing 601 may comprise mounting brackets 602, such as four mounting brackets, vertically extending from each side wall of housing 601. Mounting brackets 602 may be used to mount headwall station 600 to the structural frame of the headwall, such as between parallel structural beams. Headwall station 600 may comprise a front opening 604 to provide access to the interior space of housing 601. Headwall station 600 may further comprise opening 605 that mates with a terminal end of the pneumatic tubing 610. Headwall station 600 may further comprise a decorative frame 608 adapted to be secured about the front opening 604 of housing 601 to cover any space created between housing 601 and an opening in the headwall. The front opening 604 may be covered via door 612 such as a glass door, with similar configuration as door 112 in FIG. 1, configured for closing the front opening 604 of the housing 601 to restrict access to the headwall station 600 by unauthorized users. Upon arrival, medical objects may be directly received within the housing 601 of the headwall station 600. According to an embodiment, the interior space of the headwall station 600 may comprise a user interface unit 615 comprising various modules for interfacing with a user, such as an identifying tag reader (e.g., 115 in FIG. 1), a touch-screen (e.g., 118 in FIG. 1), a keypad (e.g., 119 in FIG. 1), a card reader 617, or the like. Although in other embodiments, the user interface unit 615 may reside outside of the interior space, as shown in FIG. 1. The user interface unit 615 may be utilized to enter various information, as discussed below, such as user identifying information, destination information, prescription information, or the like.

Headwall station 121 for disposing medical objects may comprise a similar configuration to headwall station 110 and 600. According to an embodiment, referring to FIG. 7, headwall station 121 may comprise a housing 701 adapted to be recessed in the headwall 100. Housing 701 may comprise mounting brackets 702, such as four mounting brackets, vertically extending from each side wall of housing 701. Mounting brackets 702 may be used to mount headwall station 121 to the structural frame 130 of the headwall, such as parallel structural beams. The front face of the housing 701 may include a front opening 704 for allowing access into the housing 701. Headwall station 121 may further comprise a decorative frame 708 adapted to be secured about the front opening 704 of housing 701 to cover any space created between housing 701 and an opening in the headwall. Housing 701 may further comprise opening 705 that mates with a terminal end of the pneumatic tubing 126. The opening 705 to pneumatic tubing 126 may comprise a door 712 configured for being opened to give access to the user to dispose medical objects, such as sharps, directly into the pneumatic tubing 126. Door 712 may comprise a handle 714. The headwall station 121 may comprise a door open sensor 706 that may be triggered when the door is opened 712. Door open sensor 706 may activate a pump connected to pneumatic tubing 126 such that medical objects can be quickly disposed via the headwall station 121 and transported via pneumatic tubing 126 to a disposal area as discussed below. In addition, although not shown, headwall station 121 may include similar components as headwall station 110 and 600, such as a front door 112, identifying tag reader 115, touch-screen 118, and/or a keypad 119 (FIG. 1).

Referring to FIGS. 8A-8C, there is show yet another embodiment of headwall station 800, where FIG. 8A illustrates a front perspective view of the headwall station 800, FIG. 8B illustrates a front perspective view of the headwall station 800 installed in a headwall 810, and FIG. 8C illustrates a rear perspective view of the headwall station 800 installed in the headwall 810. Headwall station 800 may incorporate both a medical object receiving and dispensing portion or bin 801 with similar construction to headwall station 110, as well as a medical object disposal portion or bin 802 with similar construction to headwall station 121. Headwall station 800 may comprise housing 804 adapted to be recessed in headwall 810 substantially behind the decorative front panel 821 of the headwall. Referring to FIGS. 8B and 8C, housing 804 may be mounted to the structural frame 820 of the headwall 810, for example via screws (not shown). The decorative front panel 821 of the headwall 810 may include an opening to provide access to the headwall station 800. A frame 815 may be used to cover any space between housing 804 and the opening in the headwall 810. Referring to FIG. 8A, housing 804 may comprise a partition 805 adapted to segregate the medical object receiving and dispensing portion or bin 801 from the medical object disposal portion or bin 802. While portions 801 and 802 are shown to be disposed horizontally or side-by-side with respect to each other, they may be also disposed vertically or one on top of the other.

The medical object disposal portion 802 may include a medical object guiding portion 807 tapering from a wider opening to a narrower opening in order to guide medical objects into pneumatic tubing 806. The medical object receiving and dispensing portion 801 may comprise a door 812 comprising a handle or knob 813. The door 812 may be closed shut via an electromagnet 814 to restrict access to portion 801 by unauthorized users. Door 812 may also comprise a spring loaded hinge 817. According to an embodiment, the medical object receiving and dispensing portion 801 may comprise a medical object arrival indicator 816, such as an LED, adapted to indicate that a medical object has arrived at headwall station 800 via pneumatic tubing 819. According to another embodiment, the medical object receiving and dispensing portion 801 (or stations 110/600) may also comprise a speaker adapted to emit an audible signal or message when a medical object and/or a carrier is received at the headwall station 800. Each of the portions 801 and 802 may further comprise an ID card reader 813 and 818, respectively, adapted to read an ID card of a user for authentication. For example, upon authentication of an ID card of a user at ID card reader 813 of the medical object disposal portion 802, a pump associated with portion 802 may be activated to dispose medical objects. On the other hand, ID card reader 818 may be used to unlock the lock 814 of door 812. According to another embodiment, headwall station 800 may comprise a user interface unit 615 shown in FIG. 6. According to an embodiment, as shown in FIG. 8C, each headwall station 800 may comprise a controller 822 in electrical communication with the headwall station 800 (or incorporated in the headwall station 800), configured for monitoring and/or controlling the operation of headwall station 800, as discussed below.

FIG. 2 shows a diagram illustrating a pneumatic tubing system 200 for distribution of medical objects 205, such as medicines, between a pneumatic station 204 and at least one headwall station 210 at a patient's room according to an embodiment of the present principles. System 200 comprises a pneumatic station 204 connected to one or more headwall stations 210 via pneumatic tubing 206. The pneumatic station 204 may be placed at a secure area within the hospital designated as a location from where medical objects are distributed to the patients, such as a nursing station or a pharmacy. According to an embodiment, the pneumatic station 204 may comprise a housing recessed in a wall within a nursing station, or another location, or it may be secured on a wall or to the floor, secured to a counter, attached to a pedestal, or installed via other means within a location. The front face of the pneumatic station 204 may include an opening in communication with an opening within the housing connected to pneumatic tubing 206. The pneumatic station 204 may further comprise a door to restrict access to the pneumatic tubing system 200. The pneumatic station 204 may contain a touch screen for user input and communication. The touch screen may allow a user to identify the intended headwall station 210 and input any other instructions for the handling of the transported medical objects 205. In an alternative embodiment, the functions of touch screen may be accomplished using manual buttons, switches or other controls, in which event a screen without touch capability may be used.

The headwall station 210 may be placed within the patient headwall 201 as discussed above with reference to FIGS. 1, 6, and 8A-8C. After prescriptions for medical objects 205, such as medicines, are filled at the pharmacy or sorted at the nursing station, they will be placed in the pneumatic station 204 and distributed via pneumatic tubing 206 to the proper headwall station 210 in proximity to the patient bedside.

According to an embodiment, system 200 is configured to operate without the implementation of any dedicated pneumatic carriers. As such, medical objects 205 may directly transported within the pneumatic tubing 206 such that no individual carriers or containers need to get loaded at the pneumatic station 204 or emptied at the headwall station 210. The medical object 205 may be distributed quickly, safely, and without prolonged handling, effectively eliminating user errors by minimizing extra staff handovers. Although according to another embodiment, medical objects 205 may be placed in a protective cylinder adapted to travel within pneumatic tubing 206. Such cylinder may be used for convenience, for more rigid restrictive processes, or where cross contamination of medical objects needs to be prevented for safety.

According to some aspects of the embodiments, pneumatic tubing 206 may comprise rigid pipes for transmitting medical objects 205 or the protective cylinders. According to other embodiments, pneumatic tubing 206 may comprise a flexible reinforced hose, such as flexible plastic material, polyvinyl chloride (PVC), polyethylene, polypropylene, or the like. Although, other materials may also be utilized, including rubber; reinforced or coated fabric, such as polyester, nylon, fiberglass, or the like; silicone; metals such as aluminum alloy, corrugated stainless steel alloy, or the like; or other material known in the art. For carrier free operation, the hose may comprise a smooth inner surface to prevent the medical objects 205 from being caught in the hose. The inner diameter of the hose may range from approximately 1 inch to approximately 4 inches. According to an embodiment, the inner diameter may comprise approximately 2 inches. Accordingly, the hose is large enough to permit the transport of medical objects 205 while maintaining small enough inner diameter to reduce the amount of air pressure or vacuum required to efficiently transport the medical objects 205 therein.

According to one embodiment, system 200 may comprise a multi-station pneumatic system comprising a pneumatic station 204 connected to a plurality of headwall stations, such as headwall stations 210 and 230, via a plurality of tubing pathways. According to an embodiment, the system 200 may comprise a diverter 231 in communication with and controlled by a system controller 212 and adapted for creating a path from the pneumatic station 204 to one of the headwall stations 210 or 230. Although FIG. 2 demonstrates a one zone pneumatic tubing system 200, a system with multiple zones and the inclusion of any number of pneumatic stations 204 and headwall stations 210 is possible without deviating from the scope of the present principles. The various pneumatic tubing pathways may be combined or merged via one or more Y-connectors or diverters configured for changing the direction of the tubing pathways. According to an embodiment, system 200 may be a one-way system such that medical objects 205 may only travel from the pneumatic station 204 to one of the headwall stations 210 in one direction, and not backward. This ensures safety of transmissions and prevents system misuse. According to other embodiments, two-way pneumatic system may be used allowing transmission of medical objects 205 to or from the headwall station 210.

System 200 may comprise at least one pump 220 configured for creating pressure differentiation within the pneumatic tubing 206 that facilitates the transmission of medical objects 205 between the pneumatic station 204 and the headwall station 210. Pump 220 may either create a positive pressure or a negative pressure within pneumatic tubing 206 to move medical objects 205, or a dedicated carrier, within the tubing 206. According to an embodiment, pump 220 may be configured for generating approximately 4 pounds to approximately 6 pounds of pressure/vacuum. Pump 220 may be connected to pneumatic tubing 206 via an airline tubing 208 configured for providing compressed air. According to one embodiment, pump 220 may comprise a positive pressure air compressor for generating compressed air within pneumatic tubing 206. In such an implementation, for example, the system 200 may comprise at least one diverter 231 and a single positive air pressure pump or air compressor 220. Such positive pressure air compressor 220 may be configured to provide positive air pressure to push medical object 205 from the pneumatic station 204 to one of the headwall stations 210 or 230 via a path created by the diverter 231. Alternatively, according to another embodiment the pump 220 may comprise a negative pressure compressor or a vacuum pump that creates vacuum within pneumatic tubing 206. In such an implementation, for example, each headwall station 210 and 230 may comprise a vacuum pump 220 that pulls the medical object 205 from the pneumatic station 204 to the respective headwall station 210 with negative air pressure. In another embodiment, pump 220 may be configured to generate positive and negative pressure within pneumatic tubing 206 to allow for a two-way transmission.

According to another aspect of the embodiments, system 200 may comprise a point-to-point pneumatic system. In such an implementation, the pneumatic station 204 may comprise a plurality of inlets 217 each connected via an independent pneumatic tubing pathway, such as pathway 219, to a respective headwall station, such as headwall station 240. Each such pneumatic tubing pathway may be associated with a pump, such as pump 241, which may be selectively activated to transmit medical objects 205 between the pneumatic station 204 and a desired headwall station.

Referring to FIG. 9, there is shown an exemplary embodiment of a pneumatic sending station 900 with a plurality of inlets 910 for distribution of medical objects to a plurality of headwall stations at the patient bedside, such as headwall stations 110, 210/230/240, 610, and/or 801. Sending station 900 may be placed at the pharmacy, at a nursing station, or a similar location from which medical objects need to be distributed to the patients. Sending station 900 may be used in a one way distribution system where medical objects need to be only sent from, but not received by, the sending station 900. Sending station 900 may comprise housing 901 adapted to contain the inlets 910. In other embodiments, sending station 900 may be implemented without a housing 901 by placing access to the inlets 910 on a counter or a similar surface. Each inlet 910 may comprise an opening 905 that mates with a terminal end of the respective pneumatic tubing 911 such as medical objects may be directly received through the opening 905 into respective pneumatic tubing 911. According to an embodiment, each inlet 910 may comprise a medical object guiding portion, such as 807 shown in FIG. 8A, that tapers from a wider opening to a narrower opening in order to guide medical objects into the respective pneumatic tubing 911. Each inlet 910 is associated with and connected via a respective pneumatic tubing 911 to a single respective headwall station such that each inlet may only deliver medical objects to an associated headwall station on the other end of the respective pneumatic tubing 911. The system consists of a plurality of pumps 918 such that each inlet 910 is also associated with a respective pump 918 connected to the respective pneumatic tubing 911 to distribute medical objects from the inlet 910 to a respective headwall station via positive or negative air pressure.

In addition, the opening 905 of each inlet 910 to respective pneumatic tubing 911 may comprise a door 912 configured for closing the opening 905 to restrict access to the inlet 910 to unauthorized users. The door 912 may comprise a biasing mechanism, such as a spring loaded hinge 913, adapted to bias the door 912 to open. The door 912 may also comprise a lock 914, such as an electromagnetic lock, that keeps the door 912 locked. Other types of locks may also be utilized. The door 912 is generally remains closed by lock 914. According to an embodiment, the sending station 901 may comprise a user interface unit 915 comprising various modules for interfacing with a user, such as an identifying tag reader (e.g., 115 in FIG. 1), a touch-screen (e.g., 118 in FIG. 1), a keypad (e.g., 119 in FIG. 1), a card reader 617, or the like. The user interface unit 915 may be utilized to enter various information, as discussed below, such as user identifying information, destination information, prescription information, or the like. According to an embodiment, the identifying tag reader of the user interface 915 may be used to scan a barcode on a medical object in order to identify the desired destination headwall station as well as its associated pump 918 and inlet 910. In response, the lock 914 of the identified inlet 910 may automatically unlock and the spring loaded hinge 913 will force the door 912 to open to provide access to the appropriate headwall station via the identified inlet 910. In addition, the identified pump 918 may be automatically turned on to distribute the medical object from the identified inlet 910 to the identified destination headwall station on the other end.

Referring back to FIG. 2, system 200 may further comprise a system controller 212 configured for monitoring and controlling the operation of system 200. The pneumatic station 204, one or more headwall stations 210, 230, and/or 240, the diverter 231, one or more pumps 220 and/or 241, and other system components, may be connected via a wired or wireless signal communication network 215 with, and controlled by, the system controller 212. Although a single system controller 212 is illustrated, a plurality of system controllers may be utilized. For example, each headwall station 210 may comprise a satellite controlling unit (e.g., controller 822 shown in FIG. 8C). According to another embodiment, the system controller 212 may be integrated within the pneumatic station 204.

In one embodiment, the system controller 212 may comprise at least one processor, such as a central processing unit (CPU), a microprocessor, a “general purpose” microprocessor, a special purpose microprocessor, an application specific integrated circuit (ASICs), general logic, or any combination thereof. The controller 212 can provide processing capability to execute an operating system, run various applications, and/or provide processing for one or more of the techniques and functions described herein. Applications that can run on the system controller 212 can include, for example, software for configuring and operating the pneumatic tubing system 200. The system controller 212 may further include a memory communicably coupled to the processor, which can store data and executable code. The memory can represent any suitable storage medium, such as volatile and/or nonvolatile memory, including random-access memory (RAM), read-only memory (ROM), Flash memory, hard disk drive, or the like. In buffering or caching data related to operations of the processor, the memory can store data associated with applications running on the system controller 212.

The system controller 212 can further comprise one or more interfaces, such as a communication network interface, an analog interface, a wireless network interface, or the like, for connecting to communication network 215. According to an embodiment, the network interface may comprise an Ethernet interface for sending and receiving signals over an Internet Protocol (IP) based network. According to one embodiment, the communication network 215 can provide a wired connection between system components. According to another embodiment, the communication network 215 can comprise a wireless network, such as an IEEE 802.11 based network or Wi-Fi.

The pneumatic station 204, and/or each headwall station 210 can comprise a memory and a processor, such as a microcontroller-based PC board, configured for communicating with and processing various commands and performing operations requested by the system controller 212. The pneumatic station 204 and each headwall station 210 can further comprise a network interface configured for bidirectional communication on the communication network 215 with the system controller 212. The network interface can comprise an analog interface, a communication network interface, a wireless interface, such as a radiofrequency transceiver, or the like.

System controller 212 may communicate with a database 213 for logging various data. The database 213 may be, for example, a relational database, a flat file database, fixed length record database, or any other data storage mechanism known or as yet undiscovered in the art. Further, the database 213 may reside on a stand-alone server, or the same machine as the system controller 212. The database 213 of the pneumatic tubing system 200 may store patient files associated with each patient, and preferably with the patient's unique ID accorded to the patient during admission to the hospital. The patient file may be created when a patient is admitted to the hospital. Each patient file may include, for example: a patient ID; name of the patient; room number of the patient; bed number of the patient; ID of the headwall station 210 associated with the patient; name, strength, diluent, and dosage of drug to be delivered to the patient; IDs of authorized users allowed access to the headwall station 210 associated with the patient, or the like. The information may be dynamic and updated by the hospital staff as required. For example, the IDs of authorized users allowed access to the headwall station 210 may change from time to time depending on the changes of shifts of hospital personnel. Database 213 may store additional information or less of the above listed information without departing from the scope of the present invention. The system controller 212 uses the above information to properly route medical object 205 from the pneumatic station 204 to a headwall station 210 associated with the patient. During delivery, the system controller 212 may also keep a log of chain of custody of the medical object 205 based on the information collected before, during, and after transportation.

The system controller 212 may interpret the data in the database 213 and generate commands in the form of signals to individual components in system 200 to control the actions of the system 200. The system controller 212 may control various components of the system 200, such as the pump 220 or a diverter 231, via relays. In another embodiment, the various components of the system 200, such as pneumatic station 204 and headwall stations 210, may comprise microprocessors configured for interpreting commands received from the system controller 212. The system controller 212 may send commands to pump 220 to activate and thereby create pressure differentiation to transport medical objects 205 through the pneumatic tubing 206. The system controller 212 may further generate and transmit commands to the diverters to change position and/or direction of the pneumatic tubing path 206 to route the medical objects 205 to the desired headwall station 210 via a particular path in the pneumatic transmission tubing 206. In another embodiment, system controller 212 may restrict access to the one or more headwall stations 210 and provide access only to authorized users.

Each pneumatic station 204 and headwall station 210 may further comprise an identifying tag reader 216. Furthermore, the pneumatic tubing 206 may include a plurality of inline identifying tag readers 218 disposed at various locations along the pneumatic tubing 206. Tag readers 216 and 218 may be configured for tracking or sensing the medical objects 205 as they are transported through the system 200. Tag readers 216 and 218 may comprise optical sensors, radiofrequency (RF) readers, or the like. Medical objects 205 may comprise identification (ID) tags 207 attached or printed on the medical objects 205, for example on a label. ID tags 207 may include, but are not limited to optically scannable identifier tag, radio-frequency identification (RFID) tags, near field communication (NFC) tags, barcodes, or similar ID tags that are capable of being read, sensed, or identified by the tag readers 216 and 218. Additionally, any other identification technology known, or as yet undiscovered, may be used within the scope of the present principles. Each ID tag 207 may comprise a unique ID number associated with the medical objects 205 or the patient. The tag readers 216 at each pneumatic station 204 and headwall stations 210 are configured for reading the ID tags 207 attached to the medical objects 205 upon departure and/or arrival. Optical inline identifying tag readers 218 may be implemented for example, through a window in a section of the tubing 206, through an optical sensor disposed in the tubing 206, or the like. Radiofrequency type identifying tag readers 218 may be implemented through a radiofrequency antenna disposed on a recess section of the tubing 206. The inline identifying tag readers 218 read, or otherwise sense, the passage of medical objects 205 comprising an ID tag 207 that is being transported through the pneumatic tubing system 200.

In one embodiment of the present principles, each tag reader 216 and 218 may be used to record information associated with the ID tags 207 attached to the medical objects 205 at various locations throughout the pneumatic tubing system 200 and send the recorded information to the system controller 212. According to another embodiment, the system 200 may utilize handheld devices, such as smartphones or personal digital assistants (PDA), for reading the ID tags 207 and transmitting recorded information to the system controller 212. The recorded information may include the ID number read from the ID tags 207 associated with the medical objects 205. The recorded information may also be appended with other relevant information, such as, but not limited to, date and time, location, a unique ID associated with the pneumatic station 204 and headwall station 210, a unique ID associated with the patient room or information identifying the room number, a unique ID associated with the user sending the medical object 205, a unique ID associated with a carrier or cylinder containing the medical object 205, or other information associated with the transmittal of the medical object 205. The one or more of the appended information may be appended by the tag readers 216 and 218, the pneumatic station 204, the headwall station 210, or the system controller 212.

The system controller 212 may receive the recorded information from the tag readers 216 at the pneumatic station 204 and headwall station 210, as well as from the inline identifying tag readers 218 disposed throughout the pneumatic tubing system 200. The system controller 212 may log the recorded information into the database 213. Using the recorded information, the system controller 212 may track each medical object's and/or carrier's location throughout the pneumatic tubing system 200 as it is sent from the pneumatic station 204, as it moves past inline identifying tag readers 218 in the pneumatic tubing 206, and as it is received at the headwall station 210, or vice versa. This creates an auditable trail indicating a chain of custody. The system controller 212 may generate records to show that medical objects 205 have been dispatched via the pneumatic station 204, received at a headwall station 210, or passed an inline identifying tag reader 218 at a certain time. Location recordation may be used to troubleshoot and initiate error notifications, such as a stuck or lost medical objects 205. Additionally, reports on chain of custody of medical objects 205 may be generated to keep record of who has dispatched the medical object 205 at the pneumatic station 204, who has received the medical object 205 at which headwall station 210, and at which specific point in time.

According to an embodiment, the ID tags 207 may also be associated with destination and intended recipient information configured for allowing automatic identification of an intended headwall station 210 associated with a patient to whom the medical object 205 is to be delivered. The destination and intended recipient information may include, but not limited to, the patient's name or identification number, a room identification number, a patient's bed identification number, and/or a headwall station identification number. The destination and intended recipient information may be stored in the ID tag 207 or in a patient file on the database 213. The tag reader 216 at the pneumatic station 204 may read the ID tags 207 on the medical object 205 and transmit the read information to the system controller 212. The system controller 212 may use the read information to determine the appropriate intended headwall station 210 associated with the destination and intended recipient information. For example, the ID tag 207 may store the headwall station ID assigned to the patient which is transmitted to the system controller 212 to identify the headwall station 210. In another example, the system controller 212 receiving the read information may query the patient file stored on the database 213 to identify the headwall station 210. For example, the ID tag 207 may store a unique identification number associated with the medical object 205, which is used by the system controller 212 to access the patient file and retrieve correlated headwall station 210 assigned to the patient. After identifying the intended headwall station 210, the system controller 212 may generate and transmit commands to the system components, for example to diverters to change position and/or direction of the pneumatic tubing path 206 to route the medical objects to the identified headwall station 210 via a particular path in the pneumatic transmission tubing 206.

According to an embodiment, the information stored and recorded by the system controller 212 may be made accessible to users via a computer 214 in communication with the system controller 212 and/or via a web browser with a remote communication device, such as a desktop computer, a laptop computer, or a handheld electronic device, such as a smartphone. In alternative embodiments, this information may be accessible via stand-alone applications, hard copy documents, or any other useful report format. A user may access the information stored and recorded by the system controller 212 to audit compliance with delivery procedures, to generate compliance reporting and manifest system documentation, to track any missing or problem deliveries, to identify or receiving notifications of system errors, such as when medical objects get stuck in pneumatic tubing 206, to manage access rights to the pneumatic system to authorized users, among other tasks.

In operation, the doctor prescribes the medical object 205, for example penicillin, to the patient and the patient file may be updated with the drug information, for example, with the name, dosage, strength, and diluent of drug to be delivered to the patient. According to an embodiment, referring to FIG. 1, prescription information may be entered at the patient's bedside at the headwall station 110 through a user interface, such as a touch screen 118 or a keypad 119. The prescription is then sent to the pharmacy to be filled out. After the prescription is filled, the medical object 205 or its container or package may be labeled with a unique ID tag 207, which upon scanning may retrieve the patient file or other information as discussed above encoded in the ID tag 207. The patient file may be updated to include the unique ID number associated with the medical object, which is stored in the unique ID tag 207. The medical object 205 may then be delivered to the pneumatic station 204.

At the pneumatic station 204, the user, such as a pharmacist or a nurse, may enter information using the touch screen at the pneumatic station 204, such as user authentication information as well as destination information. The user authentication information and/or the destination information may be transmitted to the system controller 212. In another embodiment, the front face of the pneumatic station 204 may comprise an identifying tag reader 216. The sending user may scan a user ID tag via the identifying tag reader 216 and the user identification information may be transmitted to the system controller 212 for authentication. The system controller 212 may determine whether the sending user is authorized to access the pneumatic station 204. Upon successful authentication, the sending user may then scan the ID tag 207 attached to the medical object 205 with the identifying tag reader 216 and the recorded information may be transmitted to the system controller 212. Using the unique ID tag 207 of the filled out prescription, the system controller 212 may access the patient file stored in database 213 and identify the intended headwall station 210. According to another embodiment, the identifying tag reader 216 may retrieve destination information directly from the ID tag 207 without accessing any patient file. For example, the identifying tag reader 216 may read the room number and/or the unique ID of a headwall station. Relevant delivery data received from the pneumatic station 204 may be logged in the database 213. This data may include the time the medical object 205 was sent, the ID of the pneumatic station 204, the sending user ID, and the ID tag 207 of the medical object 205, as well as other information discussed above.

According to an embodiment, in response to receiving or determining the destination information, the system controller 212 may send commands to the components of the pneumatic tubing system 200, such as system diverters, to create a path to transport the medical object 205 from the pneumatic station 204 to the intended headwall station 210. In a carrier free pneumatic system, the user may directly place the medical objects 205 in a receiving opening of the pneumatic station 204. In a carrier based pneumatic system, the user may place the medical objects 205 in a dedicated carrier or cylinder and deliver the cylinder to the pneumatic station 204.

Upon receiving the destination information from the user and/or the ID tag, and identifying the intended headwall station 210, the system controller 212 can active the pump 208. According to an embodiment, the pump 220 may be activated by pressing a button at the pneumatic station 204. According to another embodiment, the pneumatic station 204 may comprise a sensor configured for sensing that medical objects 205 or a cylinder has been placed in the pneumatic station 204, or a door open sensor configured for sensing that the door of the pneumatic station 204 has been opened. The sensor may trigger the pneumatic station 204 to send a signal to the pump 220 directing it to turn on. In another embodiment, the sensor may trigger the pneumatic station 204 to send a signal to the system controller 212, which in turn may activate the pump 220 via a power transistor and/or relay to route the medical object 205 from the pneumatic station 204 to an intended headwall station 210.

In a system with a pneumatic station 204 comprising a plurality of inlets and pumps each associated with a respective headwall station, such as sending station 901 shown in FIG. 9 and head wall station 240, the system controller 212 may identify and turn on the appropriate pump based on the received or determined destination information. For example, each room number or headwall station ID may be associated with a particular pump via for example a pump ID, which may be activated upon scanning of an ID tag 207 attached to the medical object 205. Each room number or headwall station ID may be further associated with a particular inlet and/or its associated door, which may be unlocked upon scanning of an ID tag 207 attached to the medical object 205. As such, medical objects 205 are placed in appropriate inlet for distribution.

Referring back to FIG. 2, the system controller 212 may comprise a timer configured for activating the appropriate pump, such as pump 220, for a predetermined amount of time sufficient to ensure that contents are delivered to a headwall station 210. According to another embodiment, the pump 220 is activated until the headwall station 210 indicates to the system controller 212 that the contents have been received by the headwall station 210. The headwall station 210 may comprise a proximity or motion sensor configured for detecting contents within the headwall station 210. The headwall station 210 may send the sensor trigger to the system controller 212, which in response deactivates the pump 220.

As the medical object 205 is transported within the pneumatic tubing system 200, inline tag readers 218 may scan the ID tag 207 of the medical object 205 and transmit that information to the system controller 212 to be logged as tracking information in database 213. Then, as the medical object 205 and/or carrier or a cylinder passes the identifying tag reader 216 at the headwall station 210 the medical object ID tag 207 may be automatically scanned to verify delivery. The headwall station 210 may further comprise a proximity or motion sensor that may be triggered when the medical object 205 and/or carrier or cylinder is received by the headwall station 210. The headwall station 210 may comprise a visual indicator that may light up to notify that the medical object 205 has been received. In another embodiment, the visual indicator at the headwall station 210 may be directed to turn on by the system controller 212 upon receiving delivery information from the identifying tag reader 216.

The medical object 205, such as medicine, may be released from the headwall station 210 by a receiving user when it becomes due for administering to the patient. Conveniently the medical object 205 is delivered directly to the patient's headwall 201. The receiving user may be a nurse attending the patient at the bedside of the headwall station 210. Referring to FIG. 1, the receiving user may unlock the door 112 of the headwall station 110 by entering user ID via touchscreen 118 or scanning a user ID tag via identifying tag reader 115. The user identification information may be transmitted to the system controller 212 for authentication and door unlock. The system controller 212 may look up the patient file to determine whether the receiving user is authorized to access the headwall station 110. If so, the system controller 212 sends a command to the headwall station 110 to unlock the door 112. The system controller 212 also logs relevant reception data, including, for example, the contents or medical object ID, the headwall station ID, the time the medical object arrived at the headwall station 110, the time the door 112 has been unlocked, and the receiving user identification information. Then receiving user may then deliver the medical object 205 to the patient. In that step, the receiving user may use a handheld device to scan an ID tag on the patient's wristband or other ID tag located in the proximity of the patient, as well as the ID tag of the prescription to create a log that the prescription has been delivered to the patient.

Referring to FIG. 3, there is shown a diagram illustrating a pneumatic tubing system 300 for disposing sharps from a headwall station 310 according to an embodiment of the present principles. System 300 may be configured for receiving and transporting any type of medical sharps, including but not limited to needles, syringes with and without needles, hypodermic and tubing, acupuncture needles, suture needles, tubing with needles, scalpel blades, blood vials, exposed ends of dental wires, broken glass or capillary tubes, culture dishes and slides, lancets, pipettes, root canal files, trauma scene waste that can cut, slice or pierce, or the like.

System 300 comprises one or more headwall stations, such as headwall stations 310 and 330, connected to a receiving station 304 via pneumatic tubing 306, which may comprise similar construction of pneumatic tubing 206 discussed above. The pneumatic tubing 306 may comprise a hose having smooth inner surface to prevent the sharps 305 from being caught in the hose. Each headwall station 310 is configured for receiving sharps 305 and automatically transporting the sharps 305 via pneumatic tubing 306 to the receiving station 304. Each headwall station 310 may be configured for receiving a plurality of sharps simultaneously for simultaneous transport. Conveniently, headwall station 310 may be installed directly in the patient's headwall 301, as illustrated in FIG. 1, such that routinely generated sharps waste may be immediately disposed of.

The receiving station 304 may be located in a secure area within the hospital designated as a contaminated area. According to an embodiment, the receiving station 304 may be located in proximity to a waste disposal area, such as a loading dock. The receiving station 304 may comprise a large receiving container capable of receiving vast amount of sharps 305 via multiple deliveries and from a plurality of locations. A plurality of openings may be included in the receiving container for receiving a plurality of incoming pneumatic tubing 306 connected to a plurality of headwall stations 310. After getting filled, the receiving container of the receiving station 304 may be sealed and picked up by a waste management company and replaced by another receiving container. According to another embodiment, the receiving container may be reused or may comprise a disposable container. According yet to another embodiment, the system may comprise a plurality of receiving containers, such as receiving containers 304 and 334, connected to pneumatic tubing 306. According to one embodiment, system 300 may comprise a plurality of pumps, such as pump 320, such that each receiving container may be associated with a particular pump. The system controller 312 may selectively activate a pump of a desired receiving container. In another embodiment, the system may comprise a diverter 335 connected to and controlled by the system controller 312 to route the sharps 305 to a desired receiving container. Each receiving controller may comprise a sensor or a similar device adapted to determine when the container 304/334 becomes full. The receiving containers 304 and 334 may communicate with the system controller 312 to indicate their status as being full. The system controller 312 may designate one of the containers, such as container 304 to receive disposed medical objects 305. When the designated receiving container 304 becomes full, as determined by the system controller 312, for example via the sensor, the system controller 312 may route sharps 305 to a different receiving container 334 with a status indicator of not being full. The receiving container(s) may be transported by the waste management company to a predetermined U.S. government approved location to be emptied. In another embodiment, the hospital may comprise an incinerator. The sharps 305 arriving at the receiving station 304 may be periodically disposed in the incinerator and destroyed.

According to aspects of the present embodiment, system 300 operates without the implementation of any dedicated pneumatic carriers. Instead, sharps 305 are directly transported within the pneumatic tubing 306. Accordingly, immediately after the sharps 305 are used in a procedure at the patient's bedside, the user may dispose of the sharps 305 directly via the headwall station 310 in the patient's headwall 301. The sharps 305 are then quickly transported to the receiving station 304. Since the sharps 305 are immediately disposed of without the use of any containers or carriers, there is no danger that the container or the carrier may get overfilled. Moreover, no individual carriers or containers need to get loaded at the headwall station 310 or emptied every time the receiving station 304 receives a delivery of sharps 305. Accordingly, system 300 speeds up the disposal process and effectively reduces the extent a user handles the sharps, significantly reducing the risk of accidental needle-stick injury.

Pneumatic system 300 may be implemented with similar configuration as discussed above with reference to FIG. 2. System 300 may comprise a point-to-point pneumatic system, or a multi-station pneumatic system comprising a plurality of headwall stations 310 connected to a receiving station 304 via a plurality of tubing pathways. The various pneumatic tubing pathways may be combined or merged via one or more Y-connectors or one or more diverters, such as diverter 331, configured for changing the direction of the tubing pathways. According to an embodiment, system 300 is a one-way system such that sharps 305 may only travel from the headwall stations 310 to the receiving station 304 in one direction, and not backward. This ensures safety of transmissions and prevents system misuse.

System 300 may comprises a pump 320 connected to the pneumatic tubing 306 via airline 308 and configured for creating pressure differentiation within the pneumatic tubing 306 that facilitates the transmission of sharps 305 from the headwall station 310 to the receiving station 304. Pump 320 may comprise similar configuration as pump 220 discussed above. System 300 may further comprise a system controller 312, similar to system controller 212 above, configured for monitoring, tracking, and controlling the operation of system 300. System controller 312 may communicate with a database 313 for logging various data. The one or more headwall stations 310, the receiving station 304, any diverters, the pump 320, and other system components, may be connected via a wired or wireless signal communication network 315 with, and controlled by, the system controller 312. Headwall station 310 may comprise similar configuration as pneumatic station 204 discussed above.

Pneumatic system 300 may further comprise identifying tag readers 316 at the headwall station 310 and receiving station 304 and a plurality of inline identifying tag readers 318 along pneumatic tubing 306, similar to identifying tag readers 216 and 218 discussed above, for tracking or sensing the sharps 305 as they are transported through the system 200. Sharps 305 may comprise ID tags 307 attached or printed on the sharps 305, for example on a label. Each ID tag 307 may comprise a unique ID number associated with the sharps 305. The system controller 312 may receive recorded information from the tag readers 316 and 318 and log the recorded information into the database 313 and create an auditable trail indicating a chain of custody. The information stored and recorded by the system controller 312 may be made accessible to users via a computer 314 as discussed above.

In operation, the user may open a door such as door 712 in FIG. 7, at the headwall station 310. According to an embodiment, to open the door 712, the user may enter the user's ID using a user interface (such as user interface 118 shown in FIG. 1), and the headwall station 310 may communicate the entered user's ID to the system controller 312 for authentication. In another embodiment, the sending user may scan an ID tag or an ID card associated with the sending user containing the user's ID at an ID card reader (such as 813 shown in FIG. 8A). The headwall station 310 may communicate with the system controller 312 to verify whether the user's ID belongs to an authorized user. Upon successful authentication, the system controller 312 may unlock the door 712 (or a door similar to door 112 shown in FIG. 1). The headwall station 310 may comprise a door open sensor that may cause the pump 320 to activate. The user can then drop the sharps 305 into the headwall station 310, and the sharps 305 may be substantially immediately transported to the receiving station 304 via the pressure differentiation created within the pneumatic tubing 306. Furthermore, in a system using identifying tag readers, the identifying tag readers 316 and 318 may sense, scan, or otherwise read the ID tag 307 attached to the sharps 305 and transmit recorded information to the system controller 312 for tracking purposes.

According to another embodiment, headwall station 310 may further comprise a crusher, pulverizer, or grinder configured for grinding sharps 305 that are being disposed at the headwall station 310.

Referring to FIG. 4, there is shown a diagram illustrating an automated distribution system 400 for distribution of medical objects 405, such as medicines, between a pneumatic station 404 and a headwall station 410 at a patient's room according to an embodiment of the present principles. System 400 may achieve automated distribution using a combination of a pneumatic tubing system 450 and a robot 430.

Pneumatic tubing system 450 may comprise a pneumatic station 404 connected to a robot dedicated station 432 via pneumatic tubing 406, which may comprise similar construction of pneumatic tubing 206 discussed above. The pneumatic station 404 may be placed at a secure area within the hospital designated as a location from where medical objects are distributed to the patients, such as a pharmacy. Pneumatic station 404 may comprise similar configuration as pneumatic station 204 discussed above. Pneumatic station 404 is configured for receiving medical objects 405 and automatically transporting them via pneumatic tubing 406 to the robot dedicated station 432 where a robot 430 is docked. The robot 430 retrieves the medical objects 405 and delivers them to the headwall station 410 located in the headwall 401 in the patient's room. Headwall station 410 may comprise similar configuration as station 110 shown in FIG. 1, except that it may not be connected to pneumatic tubing 106. With a combination of a pneumatic tubing system 450 and a robot 430, system 400 provides an automated and hands free operation for distribution of medical objects 405 from the pharmacy directly to the patient's headwall 401. Accordingly, the medical objects 405 may be distributed quickly, safely, and without prolonged handling, effectively eliminating user errors by minimizing extra staff handovers.

According to one embodiment, the medical objects 405 may be placed in a dedicated carrier 409 that travels within pneumatic tubing 406. According to another embodiment, pneumatic tubing system 450 may be configured to operate without the implementation of any dedicated pneumatic carriers as discussed above. As such, medical objects 405 may be directly transported within the pneumatic tubing 406 such that no individual carriers or containers need to get loaded at the pneumatic station 404 or emptied at the robot dedicated station 432.

Pneumatic tubing system 450 may be implemented with similar configuration as discussed above with reference to FIG. 2. The pneumatic tubing system 450 may comprise a point-to-point pneumatic system, or a multi-station pneumatic system comprising a plurality of robot dedicated stations 432 connected to a pneumatic station 404 via a plurality of tubing pathways. The various pneumatic tubing pathways may be combined or merged via one or more diverters configured for changing the direction of the tubing pathways. Pneumatic tubing system 450 may comprises a pump 420 connected to the pneumatic tubing 406 via airline 408 and configured for creating pressure differentiation within the pneumatic tubing 406 that facilitates the transmission of medical objects 405 and/or carrier 409 between the pneumatic station 404 and the robot dedicated station 432. Pump 420 may comprise similar configuration as pump 220 discussed above.

System 400 may further comprise a system controller 412, similar to system controller 212 above, configured for monitoring, tracking, and controlling the operation of system 400, including pneumatic tubing system 450 and robot 430. System controller 412 may communicate with a database 413 for logging various data. The pneumatic station 404, one or more robot dedicated stations 432, one or more headwall stations 410, one or more robots 430, any diverters, the pump 420, and other system components, may be connected via a wired or wireless signal communication network 415 with, and controlled by, the system controller 412. For example, the robots 430 may communicate with the system controller 412 via a wireless network 435, such as an IEEE 802.11 based network or Wi-Fi.

System 400 may further comprise identifying tag readers 416 at the pneumatic station 404, at the robot dedicated stations 432, at the robot 430, and at the headwall stations 410, as well as a plurality of inline identifying tag readers 418 along pneumatic tubing 406, similar to identifying tag readers 216 and 218 discussed above, for tracking or sensing the medical objects 405 and/or carrier 409 as they are transported through the system 400. Medical objects 405 and/or carrier 409 may comprise ID tags 407 attached or printed on the medical object 405 and/or carrier 409, for example on a label. Each ID tag 407 may comprise a unique ID number associated with the medical object 405 and/or carrier 409. The system controller 412 may receive recorded information from the tag readers 416 and 418 and log the recorded information into the database 413 and create an auditable trail indicating a chain of custody. The information stored and recorded by the system controller 412 may be made accessible to users via a computer 414 as discussed above.

In operation, the doctor may prescribe a medical object 405 to the patient and a patient file may be updated with the drug information, for example, with the name, dosage, strength, and diluent of drug to be delivered to the patient. The prescription is sent to the pharmacy to be filled out. As discussed above, the prescription may be sent using the headwall station 410. After the prescription is filled, the medical objects, or its container or package, may be labeled with a unique ID tag 407, which upon scanning may retrieve the patient file of other information encoded in the ID tag 407. The patient file may be updated to include the unique ID number associated with the medical object, which is stored in the unique ID tag 407. The medical object 405 may then be delivered to the pneumatic station 404.

At the pneumatic station 404, the user, such as a pharmacist or a nurse, may enter information using the touch screen at the pneumatic station 404, such as the destination information. The destination information may be transmitted to the system controller 412. In another embodiment, the front face of the pneumatic station 404 may comprise an identifying tag reader 416. The sending user may scan the ID tag 407 attached to the medical object 405 with the identifying tag reader 416 and the recorded information may be transmitted to the system controller 412. Using the unique ID tag 407 of the filled out prescription, the system controller 412 may access the patient file stored in database 413 and identify the intended headwall station 410. Relevant delivery data received from the pneumatic station 404 may be logged in the database 413. This data may include the time the medical object 405 was sent, the ID of the pneumatic station 404, the sending user ID, and the ID tag 407 of the medical object 405, as well as other information discussed above.

In response to receiving or determining the destination information, the system controller 412 may send commands to the components of the pneumatic tubing delivery portion of system 400, such as system diverters, to create a path to transport the medical object 405 from the pneumatic station 404 to the intended robot dedicated station 432, for example, a robot dedicated station 432 located on the same floor as the patient's room. In a carrier based pneumatic system, the user may place the medical objects 405 in a dedicated carrier 409 and deliver the carrier 407 to a carrier holder 426 at the pneumatic station 404. The ID tag 407 of the carrier 409 may be scanned by the identifying tag reader 416 at the pneumatic station 404, and the carrier identifying information may be appended to the patient file. In a carrier free pneumatic system, the user may directly place the medical objects 405 in a receiving opening of the pneumatic station 404.

Upon receiving the destination information from the user and identifying the intended robot dedicated station 432, the system controller 412 can active the pump 408. According to an embodiment, the pump 420 may be activated by pressing a button at the pneumatic station 404. According to another embodiment, the pneumatic station 404 may comprise a sensor configured for sensing that the carrier 409 was placed in the carrier holder 426. Alternatively, for example in a carrier free system, the sensor may comprise a door open sensor configured for sending that a door of the pneumatic station 404 has been opened and trigger the pump 420 to activate or turn on. The sensor may trigger the pneumatic station 404 to send a signal directly to the pump 420 directing it to turn on. In another embodiment, the signal may be send to the system controller 412, which may in turn activate the pump 420 via a power transistor and/or relay to route the medical object 405 and/or carrier 409 from the pneumatic station 404 to an intended robot dedicated station 432.

As the medical object 405 and/or carrier 409 is transported within the pneumatic tubing 406, inline tag readers 418 may scan the ID tag 407 of the medical object 405 and/or carrier 409 and transmit that information to the system controller 412 to be logged as tracking information in database 413. Then, as the medical object 405 and/or carrier 409 passes the identifying tag reader 416 at the robot dedicated headwall station 410 the medical object ID tag 407 may be automatically scanned to verify delivery.

The robot dedicated station 432 may comprise a slide gate 438, also called a slide plate. The slide gate 438 may be used for holding the medical objects 405 and/or carrier 409 in the pneumatic tubing 406 above the robot dedicated station 432 and prevent it from being delivered out of the robot dedicated station 432. The robot dedicated station 432 may comprise a sensor for indicating that a robot 430 is docked at the robot dedicated station 432. Alternatively, the robot 430 may signal to the system controller 412 that it is docked at the robot dedicated station 432 and is ready to receive medical objects 405 and/or carrier 409. If a robot 430 is indeed docked at the robot dedicated station 432 and ready to accept medical objects 405 and/or carrier 409, the robot 430 or the system controller 112 may signal the slide gate 438 to open and permit passage of the medical objects 405 and/or carrier 409 through the slide gate 438 and into the robot 430.

Robot 430 may comprise a port or opening 439 through the top surface of the robot housing configured for receiving the medical objects 405 and/or carrier 409 from the robot dedicated station 432, through the port 439, and into the robot 430. In another embodiment, the port or opening 439 may be disposed on a side of the robot 430. In a carrier based system, the robot 430 may contain one or more carrier holders or retainers 427 within its housing. In a carrier free system, medical objects 405 may be dropped into a receiving bin within the robot 435.

Robot 430 may comprise an identifying tag reader 416 in proximity to the opening 439 configured for scanning the ID tag 407 attached to the medical objects 405 and/or carrier 409 to confirm receipt by the robot 430. Recorded information may be transmitted by the robot 430 to the system controller 412 via a wireless communication network 435. In addition, using the scanned information, robot 430 may determine the destination information of the medical objects 405 and directed by logic may deliver the medical objects 405 and/or carrier 409 to the headwall station 410 at the headwall 401 of the patient's room without any intercession. Headwall station 410 may comprise similar configured to the headwall station 110 in FIG. 1 such that the robot 430 may deliver the medical objects 405 and/or carrier 409 into housing 111 recessed in the headwall 100.

The ID tag 407 on the medical objects 405 and/or carrier 409 may be scanned by an identifying tag reader 416 in the headwall station 410 to verify delivery. The medical object 405 may be released from the headwall station 410 by a receiving user when it becomes due for administering to the patient. Conveniently the medical object 405 is automatically delivered directly to the patient's headwall 401. The receiving user may be a nurse attending the patient at the bedside of the headwall station 410. Referring to FIG. 1, the receiving user may unlock the door 112 of the headwall station 410 by entering user ID via touchscreen 418 or scanning a user ID tag via identifying tag reader 415. The user identification information may be transmitted to the system controller 412 for authentication and door unlock. Then, the receiving user may deliver the medical object 405 to the patient.

Referring to FIG. 5, there is shown a rear perspective view of a robot 430 that may be used in the automated distribution system 400 according to an illustrative embodiment. Robot 430 may comprise a touch screen 502 for user input and communication. Robot 430 may comprise a port or an opening 439 through the top surface of the robot housing 510 configured for receiving the medical objects 405 and/or carrier 409 from the robot dedicated station 432. Robot 430 may comprise wheels 517 for translating along a hospital floor. Robot 430 may further comprise a delivery port 511 used to deliver medical objects 405 and/or carrier 409 to the headwall station 410.

### INDUSTRIAL APPLICABILITY

The disclosed embodiments provide a system, software, and a method for the distribution of medical objects. It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments may be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

The above-described embodiments are intended to be illustrative in all respects, rather than restrictive, of the embodiments. Thus the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

### Alternate Embodiments

Alternate embodiments may be devised without departing from the spirit or the scope of the different aspects of the embodiments.

What is claimed is:

1. A pneumatic tubing system for distribution of medical objects in a medical environment without a dedicated carrier comprising:
a plurality of headwall stations each adapted to be installed in a headwall structure proximate to a patient bedside, wherein each headwall station comprises an opening in communication with pneumatic tubing;
a sending station connected to each of the headwall stations via pneumatic tubing;
at least one pump adapted to create pressure differentiation within at least a subset of the pneumatic tubing for transmitting a medical object from the sending station to one of the headwall stations; and
at least one controller in signal communication with the sending station and the at least one pump, wherein the controller is adapted to selectively activate the at least one pump to route a medical object from the sending station to one of the headwall stations within the pneumatic tubing.

2. The pneumatic tubing system according to claim 1, wherein at least one headwall structure comprises a structural frame and a decorative front panel, wherein at least one headwall station is adapted to be recessed within the structural frame, and wherein the decorative front panel comprises an opening to provide access to the at least one headwall station.

3. The pneumatic tubing system according to claim 1, wherein at least one headwall structure comprises a headwall cabinet, wherein at least one headwall station is secured to the headwall cabinet, and wherein the headwall cabinet comprises an opening to provide access to the at least one headwall station.

4. The pneumatic tubing system according to claim 1, wherein at least one headwall station comprises a user interface, wherein the at least one headwall station comprises a door with a lock, wherein the user interface is adapted to receive user identifying information, wherein the at least one controller receives and authenticates the user identifying information from the at least one headwall station and upon successful authentication unlocks the door of the at least one headwall station.

5. The pneumatic tubing system according to claim 1, wherein at least one headwall station comprises a user interface, wherein the user interface is adapted to receive prescription information and transmit the prescription information to the sending station.

6. The pneumatic tubing system according to claim 1, wherein the sending station comprises a plurality of inlets each comprising an opening in communication with pneumatic tubing, wherein each headwall station is associated with and connected to one of the inlets via an independent pneumatic tubing path, and wherein the system further comprises a plurality of the pumps, wherein each pump is associated with one of the headwall stations and is adapted to create pressure differentiation within an associated independent pneumatic tubing path.

7. The pneumatic tubing system according to claim 6, wherein each inlet comprises a medical object guiding portion that tapers from a wider opening to a narrower opening to guide medical objects into a respective pneumatic tubing path.

8. The pneumatic tubing system according to claim 6, wherein the at least one controller is further adapted to:
receive destination information from the sending station;
identify an intended headwall station using the destination information;
identify a pump associated with the intended headwall station from the plurality of pumps; and
activate the identified pump to route a medical object from an inlet of the sending station associated with the identified headwall station to the identified headwall station.

9. The pneumatic tubing system according to claim 8, wherein each inlet comprises a door adapted to close the inlet opening, wherein each door comprises a lock adapted to keep the door locked, wherein the at least one controller is further adapted to unlock the door of the inlet associated with the identified headwall station to automatically provide access to the identified headwall station via the associated inlet.

10. The pneumatic tubing system according to claim 9, wherein the door comprises a biasing mechanism adapted to bias the door to open, wherein upon being unlocked the door of the inlet associated with the identified headwall station is forced to an open position.

11. The pneumatic tubing system according to claim 8, wherein the medical object comprises an ID tag including destination information, wherein the sending station comprises an identifying tag reader adapted to read the ID tag of the medical object to retrieve the destination information.

12. The pneumatic tubing system according to claim 8, wherein the destination information comprises at least one of a room number or ID associated with a headwall station, a unique ID of a headwall station, a unique ID of a pump associated with a headwall station, and any combinations thereof.

13. A pneumatic tubing system for distribution of medical objects in a medical environment without a dedicated carrier comprising:
a plurality of headwall stations each adapted to be installed in a headwall structure proximate to a patient bedside, wherein each headwall station comprises an opening in communication with pneumatic tubing;
a sending station comprising a plurality of inlets each associated with and connected to one of the headwall stations via an independent pneumatic tubing path;
a plurality of pumps each adapted to create pressure differentiation within at least one independent pneumatic tubing path; and
at least one controller in signal communication with the sending station and the at least one pump, wherein the controller is adapted to:
receive destination information from the sending station;
identify an intended headwall station using the destination information;
identify a pump associated with the intended headwall station from the plurality of pumps; and
activate the identified pump to route a medical object from an inlet of the sending station associated with the identified headwall station to the identified headwall station.

14. A pneumatic tubing system for disposal of medical objects in a medical environment without a dedicated carrier comprising:
a plurality of headwall stations each adapted to be installed in a headwall structure proximate to a patient bedside, wherein each headwall station comprises an opening in communication with pneumatic tubing;
a receiving container connected to each of the headwall stations via pneumatic tubing;
at least one pump adapted to create pressure differentiation within at least a subset of the pneumatic tubing for transmitting a medical object from one of the headwall stations to the receiving container; and
at least one controller in signal communication with the plurality of headwall stations and the at least one pump, wherein the controller is adapted to selectively activate the at least one pump to route a medical object from one of one of the headwall stations to the receiving container within the pneumatic tubing.

15. The pneumatic tubing system according to claim 14, wherein each headwall station comprises a medical object guiding portion tapering from a wider opening to a narrower opening in order to guide the medical object into the pneumatic tubing.

16. The pneumatic tubing system according to claim 14 further comprising a plurality of receiving containers connected to the plurality of the headwall stations via pneumatic tubing, wherein the at least one controller selectively routes the medical object from one of the headwall stations to a selected one of the receiving containers.

17. The pneumatic tubing system according to claim 16 further comprising a plurality of pumps each associated with one of the receiving containers, wherein the at least one controller selectively activates at least one pump of a desired receiving container to route the medical object from one of the headwall stations to a selected one of the receiving containers.

18. The pneumatic tubing system according to claim 16 further comprising a diverter, wherein the at least one controller is adapted to activate the diverter to route the medical object from one of the headwall stations to a selected one of the receiving containers.

19. The pneumatic tubing system according to claim 16, wherein each receiving container comprises a sensor adapted to detect when the receiving container is full, wherein the at least one controller designates a first container from the plurality of receiving containers to receive disposed medical objects and routes the medical objects to the first designated receiving container, wherein when the sensor of the first designated receiving containers indicates that the first designated receiving container is full, the at least one controller designates a second receiving container from the plurality of containers to receive disposed medical objects and routes the medical objects to the second designated receiving container.

20. The pneumatic tubing system according to claim 14, wherein at least one headwall structure comprises a structural frame and a decorative front panel, wherein at least one headwall station is recessed within the structural frame, and wherein the decorative front panel comprises an opening to provide access to the at least one headwall station.
